(19) United States
(12) Patent Application Publication
(10) Pub. No.: US 2013/0052688 A1
(43) Pub. Date: Feb. 28, 2013

(54) METHOD FOR PRODUCING SACCHARIFICATION PRE-PROCESSED MATERIAL OF LIGNOCELLULOSE-BASED BIOMASS, AND SACCHARIFICATION PRE-PROCESSING DEVICE USING SAME

(71) Applicants: Tsuyoshi Baba, Saitama (JP); Minako Onodera, Saitama (JP)

(72) Inventors: Tsuyoshi Baba, Saitama (JP); Minako Onodera, Saitama (JP)

(73) Assignee: HONDA MOTOR CO., LTD., Tokyo (JP)

(21) Appl. No.: 13/634,839

(22) PCT Filed: May 12, 2011
(86) PCT No.: PCT/JP2011/060953

§ 371 Date: Sep. 13, 2012

(30) Foreign Application Priority Data
May 12, 2010 (JP) 2010-110562

(51) Int. Cl. C12P 19/00

(52) U.S. Cl. 435/72

(57) ABSTRACT

A method for producing a saccharification pre-processed material capable of producing a saccharification pre-processed material, in which lignin is sufficiently dissociated, when lignocellulose-based biomass is pre-processed with ammonia water, and a pre-processing device used therefor are provided. A saccharification pre-processing device 1 comprises a processing means 2 for mixing a substrate with ammonia water with the concentration from 20 to 30% by mass at a mass ratio of substrate:ammonia water=1:0.7 to 1:1.3 to yield a substrate mixture; heating the substrate mixture while keeping the same at a temperature of 25 to 100° C. for 1 to 100 hours for dissociating lignin from the substrate or swelling the substrate to yield a saccharification pre-processed material containing ammonia; and separating ammonia from the saccharification pre-processed material containing ammonia to yield a saccharification pre-processed material; and an ammonia water supplying means 4 for supplying ammonia water to the processing means 2.

### TECHNICAL FIELD

The present invention relates to a method for producing a saccharification pre-processed material of lignocellulose-based biomass, and a saccharification pre-processing device used therefor.

### BACKGROUND ART

From a viewpoint of prevention of global warming, reduction of the volume of carbon dioxide emission which is believed to be one of the causes thereof has been required recently. To this end, use of a blend fuel of a liquid hydrocarbon such as gasoline and ethanol for an automobile fuel has been studied.

As such ethanol, that produced by fermentation of plant substances, e.g. farm products, such as sugarcane and corn, can be used. Since plants themselves, which are source materials of the plant substances, have absorbed carbon dioxide by photosynthesis, when ethanol originated from the plant substances are burned, the amount of emitted carbon dioxide is equal to the amount of the carbon dioxide having been absorbed by the plants themselves. In other words, the so-called carbon-neutral effect can be obtained, such that the overall emission amount of carbon dioxide becomes zero in theory.

On the other hand, there is a drawback that large scale consumption of the sugarcane or corn as a source material for ethanol would reduce the amount of food supply.

Consequently, a technique for producing ethanol using nonfood lignocellulose-based biomass as the plant substances instead of sugarcane, corn, etc. has been studied. Since the lignocellulose-based biomass contains cellulose, ethanol can be yielded by degrading the cellulose by an enzyme saccharification to a saccharide such as glucose, and fermenting the yielded saccharide. Examples of the lignocellulose-based biomass include rice straw.

Meanwhile, since the lignocellulose includes as major constituents hemicellulose and lignin in addition to cellulose and the cellulose and the hemicellulose are normally bound tightly to the lignin, an enzyme saccharification reaction with the cellulose is inhibited as it is. Consequently, for an enzyme saccharification reaction of the lignocellulose as a substrate it is desirable to dissociate lignin from the substrate in advance, or have the substrate swollen, so that the enzyme should be able to contact the substrate.

In this regard, the term “dissociate” means herein at least a part of the bonds between lignin and cellulose or hemicellulose is broken. The term “swell” means crystalline cellulose expands due to infiltration of a liquid, which generates gaps in cellulose or hemicellulose constituting the crystalline cellulose, or gaps inside a cellulose fiber.

As a result, a pre-processing device for lignocellulose-based biomass saccharification, by which lignin is physically removed from the biomass by mixing the lignocellulose-based biomass with liquid ammonia and thereafter letting the pressure drop sharply, has been known heretofore (see Patent Literature 1).

According to the conventional pre-processing device for lignocellulose-based biomass saccharification, to the lignocellulose-based biomass, liquid ammonia is added, and the obtained biomass-ammonia dispersion is heated and compressed so as not to evaporate the ammonia. Then the biomass-ammonia dispersion is discharged out of the device.

When the biomass-ammonia dispersion is discharged as above, the pressure of the biomass-ammonia dispersion is decreased rapidly, such that the liquid ammonia vaporizes and the yielded ammonia gas expands explosively. As a result, the biomass is also expanded rapidly to break bonds between the biomass and lignin physically and remove the lignin.

### CITATION LIST

### Patent Literature

Patent Literature 1: Japanese Unexamined Patent Publication No. 2005-232453

### SUMMARY OF INVENTION

### Technical Problem

However, there is a drawback with the conventional pre-processing device for lignocellulose-based biomass saccharification in that an ammonia gas separated from the biomass-ammonia dispersion must be compressed to approx. 2 MPa to be liquefied for reuse as a liquid ammonia, and therefore the cost is increased.

For eliminating the drawback, it is conceivable to use ammonia water instead of ammonia for pre-processing the lignocellulose-based biomass as a substrate. Since the ammonia water can be recovered at a normal pressure, it can be reused more easily than ammonia.

In this case, it is further conceivable that, if a substrate mixture is prepared by dispersing the lignocellulose-based biomass as a substrate in ammonia water, and the substrate mixture is heated, at least a part of bonds between lignin and cellulose or hemicellulose are chemically broken to cause dissociation. Further, it is conceivable that due to infiltration of ammonia water in the lignocellulose-based biomass, gaps are generated in cellulose or hemicellulose constituting crystalline cellulose, or gaps are generated inside a cellulose fiber, and the crystalline cellulose expands and swells.

Therefore it is desirable to set conditions suitable for pre-processing the substrate mixture prepared by dispersing the lignocellulose-based biomass as a substrate in ammonia water, to dissociate lignin sufficiently, or to swell the substrate sufficiently.

Under such circumstances, an object of the present invention is to provide a method for producing a saccharification pre-processed material of lignocellulose-based biomass capable of producing, by pre-processing a substrate mixture prepared by dispersing the lignocellulose-based biomass as a substrate in ammonia water, a saccharification pre-processed material, in which lignin is sufficiently dissociated from the substrate or the substrate is sufficiently swollen.

Another object of the present invention is to provide a saccharification pre-processing device used for the method for producing saccharification pre-processed material of lignocellulose-based biomass.

### Solution to Problem

To attain the objects, the present invention is characterized by a method for producing a saccharification pre-processed material of lignocellulose-based biomass by pre-processing lignocellulose-based biomass as a substrate prior to saccharification to yield a saccharification pre-processed material, in which lignin is dissociated from the substrate, or the substrate is swollen; the method comprising the steps of: mixing the substrate with ammonia water with the concentration in a range of 20 to 30% by mass at a mass ratio of substrate:ammonia water=1:0.7 to 1:1.3 to yield a substrate mixture; heating the substrate mixture while keeping the same at a temperature in a range of 25 to 100° C. for a time period in a range of 1 to 100 hours for dissociating lignin from the substrate or swelling the substrate to yield a saccharification pre-processed material containing ammonia; and separating ammonia from the saccharification pre-processed material containing ammonia to yield a saccharification pre-processed material.

According to a method for producing a saccharification pre-processed material of lignocellulose-based biomass of the present invention, firstly lignocellulose-based biomass as a substrate is mixed with ammonia water with the concentration in a range of 20 to 30% by mass at a mass ratio of substrate:ammonia water=1:0.7 to 1:1.3. As a result, the lignocellulose-based biomass is dispersed in the ammonia water, and a substrate mixture containing the lignocellulose-based biomass uniformly impregnated with the ammonia water can be yielded.

In this regard, if the concentration of the ammonia water is less than 20% by mass, dissociation of lignin from the substrate or swelling of the substrate becomes insufficient. Meanwhile, even if the concentration of the ammonia water exceeds 30% by mass, no further effect on dissociation of lignin from the substrate or swelling of the substrate can be obtained.

If the addition rate of the ammonia water is less than 0.7 parts by mass with respect to 1 part by mass of the substrate, the ammonia water is too little in quantity, and the substrate cannot be impregnated uniformly with the ammonia water. Consequently, dissociation of lignin from the substrate or swelling of the substrate becomes insufficient.

Meanwhile, even if the addition rate of the ammonia water exceeds 1.3 parts by mass with respect to 1 part by mass of the substrate, no further effect on dissociation of lignin from the substrate or swelling of the substrate can be obtained. Further, if the addition rate of the ammonia water exceeds 1.3 parts by mass with respect to 1 part by mass of the substrate, the energy required for heating the substrate mixture becomes too much.

Next, according to a method for producing a saccharification pre-processed material of lignocellulose-based biomass of the present invention, the substrate mixture is heated for dissociating lignin from the substrate, or swelling the substrate, to yield a saccharification pre-processed material containing ammonia water. The heating is carried out by keeping the substrate mixture at a temperature in a range of 25 to 100° C. for a time period in a range of 1 to 100 hours. As a result, lignin can be sufficiently dissociated from the substrate, or the substrate can be sufficiently swollen.

If the heating temperature is less than 25° C., the substrate mixture must be kept at the temperature for a time period beyond 100 hours in order to dissociate lignin from the substrate or swell the substrate. For this reason, the thermal energy required for dissociating lignin from the substrate, or swelling the substrate becomes too much.

Meanwhile, if the heating temperature exceeds 100° C., the retention time at the temperature required for dissociating lignin from the substrate, or swelling the substrate is less than 1 hour, and the control of the retention time becomes difficult. If the heating temperature exceeds 100° C. and the retention time exceeds an appropriate level, the substrate contained in the substrate mixture may partly stick to each other or stick to a reactor by heat, causing inconvenience.

Next, according to a method for producing a saccharification pre-processed material of lignocellulose-based biomass of the present invention, ammonia is separated from the saccharification pre-processed material containing ammonia. As a result, a saccharification pre-processed material, in which lignin is dissociated from the substrate or the substrate is swollen, and at the same time ammonia is not contained, can be yielded.

A method for producing a saccharification pre-processed material of lignocellulose-based biomass of the present invention preferably comprises, in addition to the above steps, the steps of: recovering ammonia water formed by dissolving, in water, the ammonia separated from the saccharification pre-processed material containing ammonia; and transferring the saccharification pre-processed material to a post-process step.

According to a method for producing a saccharification pre-processed material of lignocellulose-based biomass of the present invention, by dissolving in water the ammonia separated as above to form ammonia water and recovering the same, the recovered ammonia water can be easily reused. Further, according to a method for producing a saccharification pre-processed material of lignocellulose-based biomass of the present invention, by transferring the saccharification pre-processed material produced as above to a post-process step, the saccharification rate of enzyme saccharification at the post-process step can be enhanced.

Further, according to a method for producing a saccharification pre-processed material of lignocellulose-based biomass of the present invention, the substrate mixture is heated preferably by keeping the same at a temperature in a range of 60 to 90° C. for a time period in a range of 6 to 24 hours.

If the heating temperature is less than 60° C., the substrate mixture must be kept at the temperature for a time period beyond 24 hours for dissociating lignin from the substrate or swelling the substrate, and therefore the thermal energy required for dissociating lignin from the substrate or swelling the substrate may become too much. Meanwhile, if the heating temperature exceeds 90° C., the retention time at the temperature for dissociating lignin from the substrate or swelling the substrate becomes less than 6 hours, and the control of the retention time may occasionally become difficult.

A saccharification pre-processing device used for a method for producing a saccharification pre-processed material of lignocellulose-based biomass of the present invention is a saccharification pre-processing device for lignocellulose-based biomass for conducting a pre-processing prior to saccharification of lignocellulose-based biomass as a substrate to yield a saccharification pre-processed material, in which lignin is dissociated from the substrate, or the substrate is swollen; the saccharification pre-processing device comprising: a processing means for mixing the substrate with ammonia water with the concentration in a range of 20 to 30% by mass at a mass ratio of substrate:ammonia water=1:0.7 to 1:1.3 to yield a substrate mixture; heating the substrate mixture while keeping the same at a temperature in a range of 25 to 100° C. for a time period in a range of 1 to 100 hours for dissociating lignin from the substrate or swelling the substrate to yield a saccharification pre-processed material containing ammonia; and separating ammonia from the saccharification pre-processed material containing ammonia to yield a saccharification pre-processed material; and an ammonia water supplying means for supplying the ammonia water to the processing means.

According to a saccharification pre-processing device for lignocellulose-based biomass of the present invention, the step for yielding the substrate mixture, the step for yielding the saccharification pre-processed material containing ammonia, and the step for yielding the saccharification pre-processed material are conducted in a single processing means, and therefore the required thermal energy can be utilized efficiently to reduce the cost.

A saccharification pre-processing device for lignocellulose-based biomass of the present invention further preferably comprises in addition to the above constitution an ammonia water recovering means for recovering ammonia water formed by dissolving, in water, the ammonia separated from the saccharification pre-processed material containing ammonia; and a transferring means for transferring the saccharification pre-processed material to a post-process step.

If a saccharification pre-processing device for lignocellulose-based biomass of the present invention comprises the ammonia water recovering means, the ammonia separated as above can be easily recovered by being dissolved in water to form ammonia water. Further, if a saccharification pre-processing device for lignocellulose-based biomass of the present invention comprises the transferring means, the saccharification pre-processed material produced as above can be transferred to a post-process step for enzymatic saccharification at the post-process step.

A saccharification pre-processing device for lignocellulose-based biomass of the present invention preferably comprises, in addition to the above components, an ammonia water recycling means for recycling the ammonia water recovered by the ammonia water recovering means to the ammonia water supplying means; and an ammonia water concentration regulating means for regulating the concentration of the ammonia water to be recycled to the ammonia water supplying means.

If a saccharification pre-processing device for lignocellulose-based biomass of the present invention comprises the ammonia water recycling means, the ammonia water recovered by the ammonia water recovering means can be supplied to the ammonia water supplying means for reuse. In this case, the concentration of the ammonia water recovered by the ammonia water recovering means may occasionally not reach the concentration range for the ammonia water to be added to the lignocellulose-based biomass. For such event, if the saccharification pre-processing device for lignocellulose-based biomass of the present invention comprises the ammonia water concentration regulating means, the concentration of the recovered ammonia water can be regulated to a predetermined concentration range.

### BRIEF DESCRIPTION OF DRAWINGS

FIG. 1 is a system block diagram showing an example of a constitution of a saccharification pre-processing device for lignocellulose-based biomass of the present invention.

FIG. 2 is a graph showing the relationship between the concentration of ammonia water added to a substrate at saccharification pre-processing and the saccharification rate at enzyme saccharification.

FIG. 3 is a graph showing the relationship between the mass of ammonia water added to 1 part by mass of a substrate at saccharification pre-processing and the saccharification rate at enzyme saccharification.

FIG. 4 is a graph showing the relationship between the retention time, when a substrate mixture is heated at a temperature of 80° C., 100° C., and 120° C. at saccharification pre-processing, and the saccharification rate at enzyme saccharification.

FIG. 5 is a graph showing the relationship between the retention time, when a substrate mixture is heated at a temperature of 25° C., 50° C., 60° C., 80° C., and 100° C. at saccharification pre-processing, and the saccharification rate at enzyme saccharification.

### DESCRIPTION OF EMBODIMENT

An embodiment of the present invention will be described below in more detail referring to appended drawings.

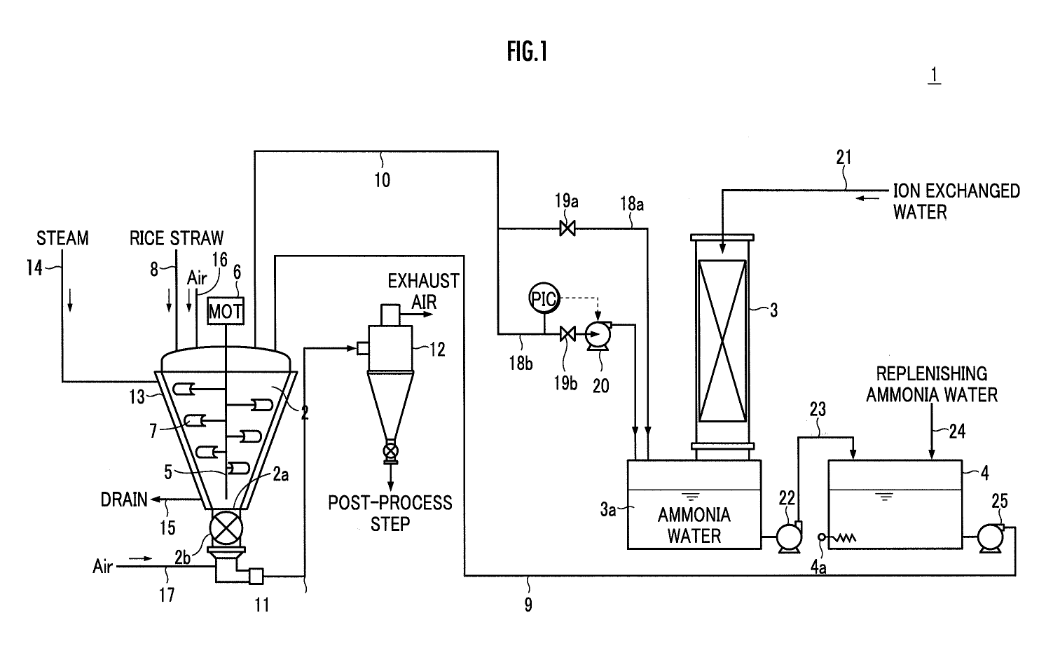

According to a method for producing a saccharification pre-processed material of lignocellulose-based biomass of the current embodiment, a saccharification pre-processed material of lignocellulose-based biomass is produced by a saccharification pre-processing device 1 shown in FIG. 1.

The saccharification pre-processing device 1 is provided with a reaction vessel 2 as a processing means, an absorber 3 as an ammonia water recovering means, and an ammonia water tank 4 as an ammonia water supplying means.

In the reaction vessel 2, lignocellulose-based biomass as a substrate and ammonia water are mixed to yield a substrate mixture, which is kept at a predetermined temperature for a predetermined time period to yield a saccharification pre-processed material containing ammonia. Then ammonia is separated by evaporation from the saccharification pre-processed material containing ammonia to yield a saccharification pre-processed material not containing ammonia.

In the absorber 3, the ammonia evaporated from the saccharification pre-processed material yielded in the reaction vessel 2 is absorbed by water and recovered as ammonia water. In the ammonia water tank 4, the ammonia water obtained in the absorber 3 is stored to be supplied to the reaction vessel 2.

The reaction vessel 2 is a vessel formed in an inverted conical shape, in which such processing as stirring of the substrate and ammonia water, heating of the substrate mixture, and evaporation of ammonia from the saccharification pre-processed material containing ammonia are conducted. For this purpose, the reaction vessel 2 is equipped with a vertical shaft 5 hung inward and a motor 6 placed in the upper part for rotating and driving the vertical shaft 5, and the vertical shaft 5 is provided with agitator blades 7 extending horizontally.

In the upper part of the reaction vessel 2, there are provided a substrate supply pipe 8 for supplying lignocellulose-based biomass as a substrate, an ammonia water supply pipe 9, and an ammonia gas pipe 10. The ammonia water supply pipe 9 is connected with the ammonia water tank 4, and the ammonia water supplied from the ammonia water tank 4 is led to the reaction vessel 2. The ammonia gas pipe 10 is connected with the absorber 3 and the ammonia gas generated in the reaction vessel 2 is sent out to the absorber 3.

In the lower part of the reaction vessel 2, there is provided an outlet 2a for discharging a saccharification pre-processed material. The outlet 2a is connected via a shut-off damper 2b with a transfer pipe 11 as a transferring means for transferring the saccharification pre-processed material to a post-process step, and the other end of the transfer pipe 11 is connected with a cyclone 12.

On the outer surface of the reaction vessel 2, there is provided a jacket 13 for regulating the internal temperature. The jacket 13 can regulate the internal temperature of the reaction vessel 2 by circulating steam internally, and for this purpose is connected with a steam supply pipe 14 for supplying steam in the upper part, and a drainage pipe 15 in the lower part.

Further, the reaction vessel 2 is provided with a 1st air supply pipe 16 for supplying pressurized air into the reaction vessel 2, and a 2nd air supply pipe 17 for supplying pressurized air to the transfer pipe 11. The 1st air supply pipe 16 is connected with the upper part of the reaction vessel 2 and the 2nd air supply pipe 17 is connected with the end of the transfer pipe 11 on the side of the shut-off damper 2b.

The ammonia gas pipe 10 provided in the upper part of the reaction vessel 2 is bifurcated to a 1st gas discharge pipe 18a and a 2nd gas discharge pipe 18b between the reaction vessel 2 and the absorber 3. The 1st gas discharge pipe 18a is provided with a shut-off valve 19a in the middle. The 2nd gas discharge pipe 18b is provided with a shut-off valve 19b in the middle, as well as a vacuum pump 20 downstream of the shut-off valve 19b.

The absorber 3 is provided with an ammonia water receiver 3a in the lower part, and an ion exchanged water supply pipe 21 in the upper part. The ammonia water receiver 3a is connected with a liquid pipe 22 as an ammonia water recycling means, and the liquid pipe 22 is connected with the ammonia water tank 4 via a pump 23.

The ammonia water tank 4 is provided with an ammonia concentration sensor and a concentrated ammonia water supply pipe 24 as ammonia water concentration regulating means. In the lower part of the ammonia water tank 4 is connected with the ammonia water supply pipe 9, which is equipped halfway with a pump 25.

The operation of the saccharification pre-processing device 1 of the current embodiment will be described below.

In the saccharification pre-processing device 1 of the current embodiment, rice straw that is lignocellulose-based biomass is supplied as a substrate to the reaction vessel 2 through the substrate supply pipe 8, and ammonia water is also supplied to the reaction vessel 2 through the ammonia water supply pipe 9. According to the current embodiment, a 5 to 35% by mass ammonia water is supplied to the reaction vessel 2 at a mass ratio in a range of 0.7 to 1.3 parts by mass with respect to 1 part by mass of the rice straw. Then, driving the motor 6 to rotate the agitator blades 7 for stirring the rice straw and the ammonia water to yield a substrate mixture in which the rice straw and the ammonia water are mixed.

According to the current embodiment, rice straw as the substrate is pulverized by a cutter mill such that at least particles with the particle size of 1 mm or larger occupy cumulatively 30% or higher. The rice straw pulverized as above can yield the substrate mixture by stirring with the ammonia water in the reaction vessel 2 at a low rotation speed for a short time without causing coagulation of the rice straw. If the rice straw is crushed finer than the above range, and stirred with ammonia water, the finely crushed rice straw may coagulate to a clayey state, which is difficult to stir.

Next, the substrate mixture in the reaction vessel 2 is heated while keeping the same at a predetermined temperature, for example, a temperature in a range of 25 to 100° C., preferably in a range of 60 to 90° C. for a time period in a range of 1 to 100 hours, preferably in a range of 6 to 24 hours. The heating of the substrate mixture is carried out, for example, by keeping the same at a temperature of 60° C. for 24 hours, or at a temperature of 80° C. for 8 hours. The heating may be conducted by supplying steam to the jacket 13 via the steam supply pipe 14.

As a result, a saccharification pre-processed material containing ammonia may be yielded, in which lignin is dissociated from a substrate containing lignin tightly bound to cellulose or hemicellulose, or the substrate is swollen. By dissociating lignin from the substrate, or swelling the substrate as above, the cellulose or hemicellulose in the substrate can be saccharified enzymatically at a post-process step.

If the substrate mixture in the reaction vessel 2 is heated as above, when the saccharification pre-processed material containing ammonia is yielded, the inside of the reaction vessel 2 is pressurized and the ammonia gas contained in the saccharification pre-processed material containing ammonia is about to evaporate by itself. Therefore, subsequently, the ammonia gas is allowed to evaporate out of the saccharification pre-processed material containing ammonia in the reaction vessel 2. Since at the start of the evaporation, the inside of the reaction vessel 2 is in a pressurized state, the shut-off valve 19a in the 1st gas discharge pipe 18a is opened and at the same time the shut-off valve 19b in the 2nd gas discharge pipe 18b is closed to send out the ammonia gas to the absorber 3 via the 1st gas discharge pipe 18a.

If the ammonia gas is sent out as above, the pressure inside the reaction vessel 2 decreases with time and the evaporation rate of the ammonia gas decreases too. Therefore, if the evaporation rate of the ammonia gas decreases below the predetermined value, the shut-off valve 19a in the 1st gas discharge pipe 18a is closed, and the shut-off valve 19b in the 2nd gas discharge pipe 18b is opened and the vacuum pump 20 is driven. In this way, the ammonia gas can be further sent out to the absorber 3 via the 2nd gas discharge pipe 18b. As a result, the ammonia is sufficiently evaporated from the saccharification pre-processed material containing ammonia, and a saccharification pre-processed material from which ammonia has been removed can be yielded.

Since the rice straw as a substrate is pulverized as above, the ammonia can be sufficiently evaporated from the saccharification pre-processed material containing ammonia, and the amount of ammonia remained in the saccharification pre-processed material can be decreased. On the other hand, if the rice straw as a substrate is pulverized finer than the above range and mixed with ammonia water to a clayey state, the ammonia may remain inside the clayey rice straw and not evaporated sufficiently.

Next, the water content of the saccharification pre-processed material from which ammonia has been removed is regulated in the reaction vessel 2. The water content can be regulated by supplying steam to the jacket 13 via the steam supply pipe 14 to heat the inside of the reaction vessel 2 for a predetermined time period. As the consequence thereof, a saccharification pre-processed material that can be sent by powder transportation can be yielded.

Next, by opening the shut-off damper 2b of the reaction vessel 2, and also supplying pressurized air from the upper part of the reaction vessel 2 via the 1st air supply pipe 16, the saccharification pre-processed material is discharged from the outlet 2a. On this occasion, pressurized air is supplied to the transfer pipe 11 via the 2nd air supply pipe 17 to transfer the saccharification pre-processed material discharged from the outlet 2a via the transfer pipe 11 to the cyclone 12.

The saccharification pre-processed material is separated from exhaust air by the cyclone 12, and then transferred further to a post-process step.

Meanwhile, the ammonia gas separated from the saccharification pre-processed material containing ammonia in the reaction vessel 2 is supplied to the absorber 3. The ammonia gas is absorbed by ion exchanged water sprayed via an ion exchanged water supply pipe 21 from the upper part of the absorber 3, and recovered as ammonia water to be stored in the ammonia water receiver 3a. The ammonia water recovered as above is sent from the ammonia water receiver 3a by the liquid pipe 22 and the pump 23 to the ammonia water tank 4.

The concentration of the ammonia water sent to the ammonia water tank 4 is adjusted to 5 to 35% by mass with concentrated ammonia water supplied via the concentrated ammonia water supply pipe 24 corresponding to an ammonia concentration detected by the ammonia concentration sensor. The ammonia water adjusted to the concentration is supplied to the reaction vessel 2 via the ammonia water supply pipe 9 to be reused for mixing with biomass.

Next, an example of a method for producing saccharification pre-processed material of lignocellulose-based biomass using the saccharification pre-processing device 1 shown in FIG. 1 will be described.

In the current embodiment, first the rice straw as a substrate and the ammonia water were supplied to the reaction vessel 2 at a mass ratio of rice straw:ammonia water=1:1 to yield a substrate mixture. The concentration of the ammonia water was varied in a range of 30% by mass or less. Next, the substrate mixture was heated and kept at a temperature of 80° C. for 8 hours in the reaction vessel 2, to yield a saccharification pre-processed material containing ammonia, in which lignin was dissociated from the substrate, or the substrate was swollen.

Next, the ammonia gas was evaporated from the saccharification pre-processed material containing ammonia in the reaction vessel 2 to yield a saccharification pre-processed material.

Next, the saccharification pre-processed material was transferred via the transfer pipe 11 to the cyclone 12, and further from the cyclone 12 to an enzyme saccharification step as a post-process step.

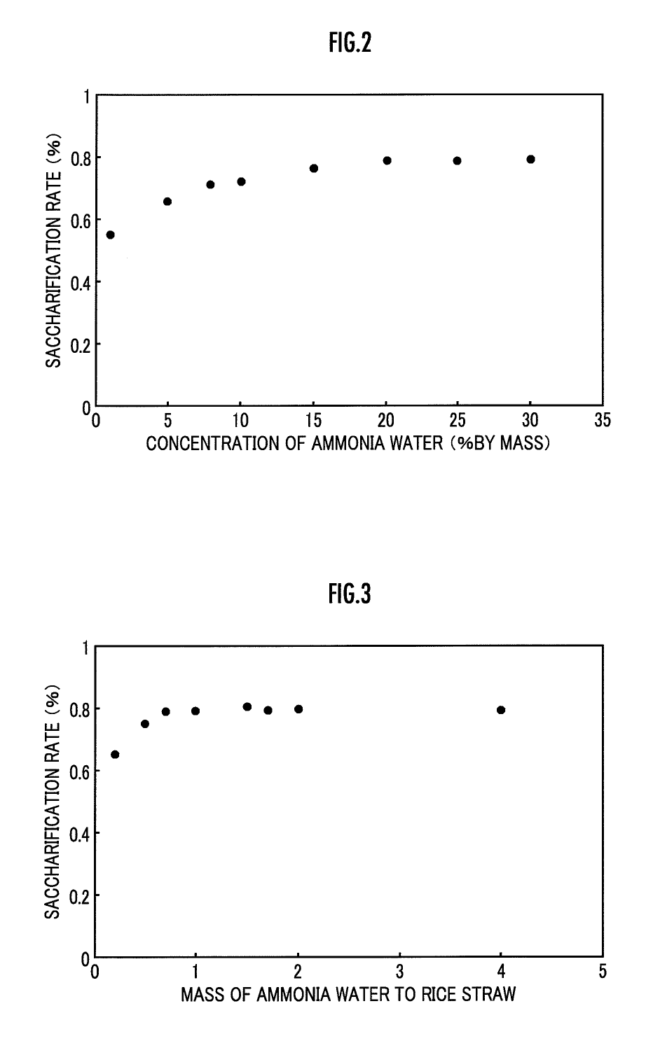

Next, the relationship between the concentration of the ammonia water in the method for producing saccharification pre-processed material and the saccharification rate at the enzyme saccharification step is shown in FIG. 2. The saccharification rate is an indicator of the condition of the dissociation of lignin from the substrate or the swelling of the substrate, and the higher saccharification rate means the better condition of the dissociation of lignin from the substrate or the swelling of the substrate.

In FIG. 2, in a concentration range below 20% by mass, the higher the concentration of the ammonia water is, the higher the saccharification rate becomes; however in a range of 20 to 30% by mass the saccharification rate is almost constant. It is clear therefore that by setting the concentration of the ammonia water to be in the range of 20 to 30% by mass, lignin can be sufficiently dissociated from the substrate or the substrate can be sufficiently swollen.

Next the relationship between the quantity of a 25% by mass concentration ammonia water to be added to 1 part by mass of the rice straw as a substrate in the method for producing a saccharification pre-processed material and the saccharification rate at the enzyme saccharification step is shown in FIG. 3.

In FIG. 3, in a range where the ammonia water is less than 0.7 parts by mass with respect to 1 part by mass of the rice straw, if the ammonia water quantity increases, the saccharification rate also increases; however in a range where the ammonia water is 0.7 parts by mass or more, the saccharification rate is almost constant. It is clear therefore that by setting the ammonia water quantity to be in a range of 0.7 to 1.3 parts by mass with respect to 1 part by mass of the rice straw, lignin can be sufficiently dissociated from the substrate or the substrate can be sufficiently swollen, and that a further effect cannot be obtained beyond 1.3 parts by mass.

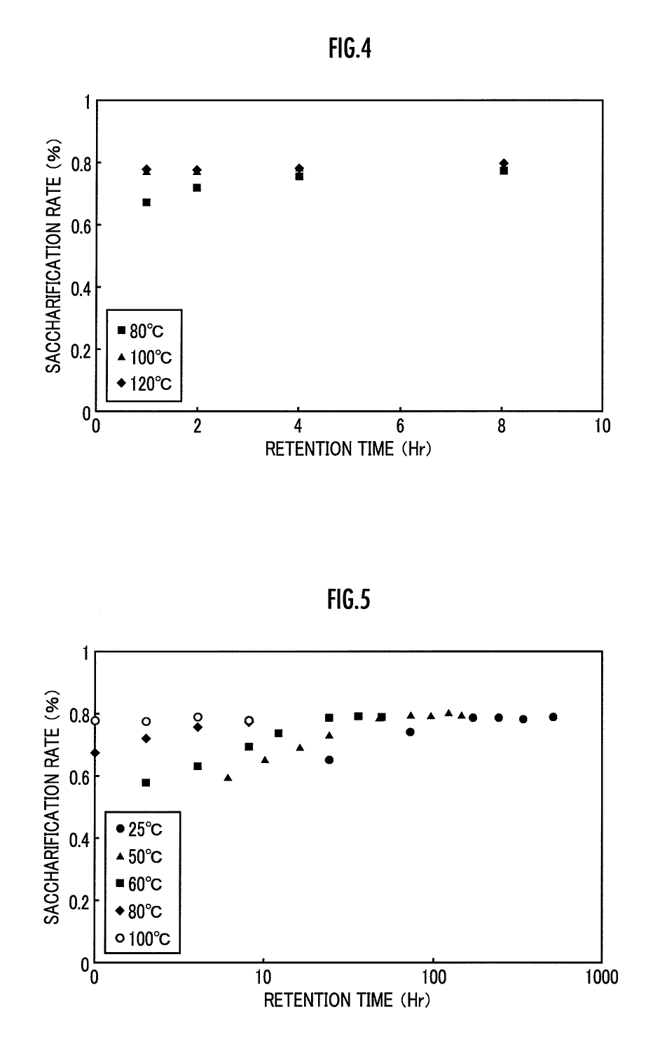

Next, the relationship between the retention time, during which the substrate mixture obtained by adding the 25% by mass concentration ammonia water to the rice straw at a mass ratio of 1:1 is heated according to the method for producing saccharification pre-processed material, and the saccharification rate at enzyme saccharification is shown in FIG. 4 and FIG. 5. FIG. 4 shows the cases of heating temperatures of 80° C., 100° C., and 120° C., and FIG. 5 shows the cases of heating temperatures of 25° C., 50° C., 60° C., 80° C., and 100° C.

In FIG. 4, if the heating temperature is in a range of 80 to 120° C., after the retention time of 8 hours at each temperature, the saccharification rate is saturated, and there is little difference between 100° C. and 120° C. In FIG. 5, if the heating temperature is 25° C., the saccharification rate is saturated after 100 hours, and at 100° C. it is saturated after 1 hour, while in a range of 50 to 80° C. the saccharification rate is saturated after a time period in a range of 1 to 100 hours depending on each temperature.

It is clear therefore that, if the substrate mixture is kept at a temperature in a range of 25 to 100° C. for a time period in a range of 1 to 100 hours, lignin can be sufficiently dissociated from the substrate, or the substrate can be sufficiently swollen.

### REFERENCE SIGNS LIST

1: saccharification pre-processing device for lignocellulose-based biomass, 2: reaction vessel (processing means), 3: scrubber (ammonia water recovering means), 4: ammonia water tank (ammonia water supplying means), 4a: ammonia concentration sensor (ammonia water concentration regulating means), 11: transfer pipe (transferring means), 22: liquid pipe (ammonia water recycling means), 24: concentrated ammonia water supply pipe (ammonia water concentration regulating means).

1. A method for producing a saccharification pre-processed material of lignocellulose-based biomass by pre-processing lignocellulose-based biomass as a substrate prior to saccharification to yield a saccharification pre-processed material, in which lignin is dissociated from the substrate, or the substrate is swollen; the method comprising the steps of:
mixing the substrate with ammonia water with the concentration in a range of 20 to 30% by mass at a mass ratio of substrate:ammonia water=1:0.7 to 1:1.3 to yield a substrate mixture;
heating the substrate mixture while keeping the same at a temperature in a range of 25 80 to 100° C. for a time period in a range of 1 to 100 hours for dissociating lignin from the substrate or swelling the substrate to yield a saccharification pre-processed material containing ammonia; and
separating ammonia gas from the saccharification pre-processed material containing ammonia to yield a saccharification pre-processed material.

2. The method for producing a saccharification pre-processed material of lignocellulose-based biomass according to claim 1, further comprising the steps of: recovering ammonia water formed by dissolving, in water, the ammonia gas separated from the saccharification pre-processed material containing ammonia; and transferring the saccharification pre-processed material to a post-process step.

3. (canceled)

4. A saccharification pre-processing device for lignocellulose-based biomass for conducting a pre-processing prior to saccharification of lignocellulose-based biomass as a substrate to yield a saccharification pre-processed material, in which lignin is dissociated from the substrate, or the substrate is swollen; the saccharification pre-processing device comprising:
a processing means for mixing the substrate with ammonia water with the concentration in a range of 20 to 30% by mass are mixed at a mass ratio of substrate:ammonia water=1:0.7 to 1:1.3 to yield a substrate mixture; heating the yielded substrate mixture and keeping at a temperature in a range of 25 80 to 100° C. for a time period in a range of 1 to 100 hours for dissociating lignin from the substrate or swelling the substrate to yield a saccharification pre-processed material containing ammonia; and separating ammonia gas from the yielded saccharification pre-processed material containing ammonia to yield a saccharification pre-processed material; and
an ammonia water supplying means for supplying the ammonia water to the processing means.

5. The saccharification pre-processing device for lignocellulose-based biomass according to claim 4, further comprising: an ammonia water recovering means for recovering ammonia water formed by dissolving, in water, the ammonia gas separated from the substrate mixture containing ammonia; and
a transferring means for transferring the saccharification pre-processed material to a post-process step.

6. The saccharification pre-processing device for lignocellulose-based biomass according to claim 5, further comprising: an ammonia water recycling means for recycling the ammonia water recovered by the ammonia water recovering means to the ammonia water supplying means; and an ammonia water concentration regulating means for regulating the concentration of the ammonia water to be recycled to the ammonia water supplying means.
